(19) United States
(12) Patent Application Publication
(10) Pub. No.: US 2005/0276765 A1
(43) Pub. Date: Dec. 15, 2005

(54) Preventing skin damage

(76) Inventors: Paul Nghiem, Newton, MA (US); Seishiro Fujii, Boston, MA (US)

Correspondence Address: FISH & RICHARDSON PC, P.O. BOX 1022, MINNEAPOLIS, MN, 55440-1022, US

(21) Appl. No.: 11/149,126

(22) Filed: Jun. 9, 2005

Related U.S. Application Data
(60) Provisional application No. 60/578,799, filed on Jun. 10, 2004.

(51) Int. Cl. A61K 7/42; A61K 31/522

(52) U.S. Cl. 424/59; 514/263.34

(57) ABSTRACT

Disclosed is, inter alia, a method of reducing UVB-induced wrinkles in a subject, the method that includes: administering to a subject having, or at risk for, UVB-induced wrinkle, a composition comprising an agent that inhibits ATR mediated signaling.

### CROSS-REFERENCE TO RELATED APPLICATIONS

This utility application claims the benefit of priority of U.S. Application Ser. No. 60/578,799, filed on Jun. 10, 2004, under 35 U.S.C. § 119. The contents of this application is hereby incorporated by reference in its entirety.

### BACKGROUND

Exposure to ultraviolet-B (UVB) irradiation can result in wrinkling of the skin.

### SUMMARY

Inhibiting ATR (a protein kinase involved in DNA replication checkpoint) and/or inhibiting the ATR-mediated replication checkpoint cascade can reduce skin damage, for example, UV-induced skin damage, e.g., wrinkles. In particular, inhibition of ATR can reduce UVB-induced wrinkles.

Accordingly, in one aspect, this disclosure features a method of treating a subject. The method includes (a) identifying a subject at risk for or having skin damage, for example, skin photodamage (e.g., wrinkles) due to UVB-exposure; and (b) administering to the subject an agent that modulates ATR signaling in the subject, e.g., administering to the subject an effective amount of an agent that decreases the activity, level or expression of ATR, e.g., an agent described herein. For example, the subject is a human subject. Preferably, the agent is administered to the subject's skin, e.g., topically. In a preferred embodiment, UVB-induced wrinkles of the skin are prevented or reduced.

In a preferred embodiment, the subject will be, is, or has been, exposed to chronic UVB radiation. UVB-radiation includes natural sunlight or artificial UVB radiation (e.g., a UVB sun lamp, e.g., for tanning, or for phototherapy, e.g., for treatment of psoriasis, atopic dermatitis, or vitiligo). The exposure is preferably for a time and in an amount sufficient to cause wrinkles. For example, chronic exposure can be exposure to the sun at a UV index of 3-6, or higher, for at least 10 minutes at least 3, more preferably at least 5, or at least 10 times in a preselected period of time. The preselected period of time can be 1 month, 2 months, 3 months, 6 months, 12 months or 24 months, e.g., exposure to a cumulative 5 hours of UVB radiation, e.g., sunlight or artificial UVB radiation, in a 12 month period. A subject at risk of chronic UV-induced wrinkles can be a subject who has been, or will be, exposed to at least 10 minutes of sun at a UV index of 3-6, or higher, at least 10 times during a one year period, or a subject who has been or will be exposed to a cumulative 5 hours of UVB radiation in one year. Preferably, the subject is exposed to at least 30 minutes of UVB radiation at least 20 times a year for at least 3 years. Preferably, the subject is exposed to the sun between 11 A.M. and 3 P.M., or the subject is exposed to the sun during the summer months, or the subject is exposed to the sun on days of high to extreme UV index. A subject at risk for long term UVB induced skin damage, e.g., wrinkles, includes: a person who lives at a high altitude, e.g., a person who lives at least 11000 feet above sea level; a person who lives near the equator, e.g., within 1000 miles from the equator; a person who uses indoor tanning parlors, e.g., at least once, 5, 10, 15, or 20 times a year, a person who participates in an outdoor activity, such as outdoor sports, at least 10, 20, 50, 80, or 100 times in one year, e.g., a person who participates in jogging, playing tennis, mountain climbing; snow skiing, water skiing, merely lying on the sands of a pleasant beach or sunning by the pool; and a person who is undergoing or has undergone UVB phototherapy. In a preferred embodiment the subject is at least 5 years of age. Preferably, the subject is at least 10, 15, 20, 25, 30, 35, 40, 45, 50, or more years of age.

In a preferred embodiment, the agent is administered using a carrier, e.g., via a liposome carrier, e.g., a lecithin liposome or an alkylphospholipid liposome. In one embodiment, the agent is formulated, e.g., as a moist paste, lotion, gel, salve, cream, or ointment, e.g., a composition that includes water but that is not in a completely liquid state. For example, the agent is formulated as a composition with a viscosity less than 15,000, 12,000, or 10,000 cP. A lotion can also include a pharmaceutically-acceptable oil phase emulsified with one or more surfactants. The agent can be administered to the face, chest, neck, hands, and other regions of the body. The treatment can involve more than one administration, e.g., at least two, three, or four administrations, of the agent. In certain cases, the site of administration is free of a skin cancer or skin tumor, e.g., a malignant or non-malignant keratosis or epithelial carcinoma. The treatment can also involve daily administration of the agent, or multiple administrations within a day, e.g., if the subject is under conditions requiring such administration, e.g., greater than moderate, or high to extreme sun or other UVB exposure.

In one embodiment, the method includes administering the agent in combination with a second treatment, e.g., a second treatment for skin, e.g., a sunscreen, tanning agent, antibiotic or moisturizer. For example, administering the agent in combination can include administering a formulation (e.g., for topical administration) that includes both the agent and a second agent that also provides a treatment for skin, e.g., for UVB induced skin damage. In certain implementations, the formulation is substantially free of catechins, e.g., free of epigallocatechin gallate (EGCG) or includes less than 6.5 or 3 mol of EGCG In some embodiments, the agent is administered to the subject in combination with a controlled release device, e.g., a patch, biocompatible polymer, micro particle, or mesh. The device may reduce degradation and control the release of the agent.

In some embodiments, the method includes evaluating the subject for wrinkles. The evaluation can be performed before, during, and/or after the administration of the agent. For example, the evaluation can be performed at least 4 hours, 8 hours, 12 hours, 1 day, 2 days, 4, 7, 14, or more days before and/or after the administration. The evaluation of wrinkles can be qualitative or quantitative. In either case, the evaluation typically includes comparing wrinkles in a treated region of the body with a reference. The reference can be, e.g., an untreated area of the subject's body, the treated area of the body as it was documented before exposure to UV and/or before treatment, an area of the subject's body not exposed to the same level of UV as the treated area, or the skin of an age-matched control subject.

In a preferred embodiment, the administration of an agent can be performed: prior to exposure to UVB light, e.g., prior to sun exposure; when UVB induced skin damage (e.g., a wrinkle) is noticed or diagnosed; at the time a treatment for wrinkles is begun or begins to exert its effects; or generally, as is needed to maintain skin health. In a preferred embodiment, the agent is administered chronically. In a preferred embodiment, the agent is administered at least once a week, preferably 2, 3, 4, 5 times a week or daily for at least two weeks, preferably for at least 1, 2, 3, 4, 5, 6 months, 1 year, 2 years or more. For example, the agent is administered periodically over 3-12 weeks, e.g., it is administered throughout the summer. In a preferred embodiment, the agent is administered to, and wrinkles are reduced or prevented on, one or more of: the subject's face, neck, chest, ears, hands, bald spots of the scalp, or any other skin that is exposed to UVB radiation.

The period over which the agent is administered, or the period over which clinically effective levels are maintained in the subject, can be short term, e.g., for one day, two days, one week, or long term, e.g., for six months or more or a year or more.

The identification of a subject in need of treatment for wrinkles can be performed e.g., by the subject, by a health care provider, by a provider of a wrinkle treatment, or by another party. The agent may be administered, e.g., by the subject, by a health care provider, by a provider of a wrinkle treatment, or another party. Likewise, the evaluation of the effect on wrinkles may be performed, e.g., by the subject, by a health care provider, by a provider of a wrinkle treatment, or another party.

An agent that decreases ATR signaling to thereby decrease UVB-induced wrinkles can be, for example: an ATR-binding protein, e.g., a soluble ATR-binding protein that binds and inhibits ATR activity, or inhibits the ability of ATR to interact with a binding partner (e.g., with ATRIP); an antibody that specifically binds to ATR, e.g., an antibody that disrupts ATR's ability to bind to a binding partner; a mutated inactive ATR or fragment thereof that binds to ATR but disrupts ATR signaling; an ATR nucleic acid molecule that can bind to a cellular ATR nucleic acid sequence, e.g., mRNA, and can inhibit expression of the protein, e.g., an antisense, siRNA molecule or ribozyme; an agent that decreases ATR gene expression, e.g., a small molecule that binds the promoter of ATR; or a crude or semi-purified extract, e.g., a botanical extract such as a plant extract, or algal extract. In another preferred embodiment, ATR is inhibited by decreasing the level of expression of an endogenous ATR gene, e.g., by decreasing transcription of the ATR gene. In a preferred embodiment, transcription of the ATR gene can be decreased by: altering the regulatory sequences of the endogenous ATR gene, e.g., by the addition of a negative regulatory sequence, such as a DNA-binding site for a transcriptional repressor, or by the removal of a positive regulatory sequence, such as an enhancer or a DNA-binding site for a transcriptional activator. In another preferred embodiment, the antibody that binds ATR is a monospecific antibody, e.g., a monoclonal antibody, e.g., a humanized, chimeric or human monoclonal antibody.

In a preferred embodiment, the agent that decreases ATR signaling, e.g., ATR activity, expression or levels is a xanthine, e.g., caffeine or a xanthine other than caffeine, e.g., theophylline or theobromine or other methyl xanthine or dimethyl xanthine. Although caffeine is a preferred agent, any suitable agent (e.g., an agent other than a xanthine) that decreases ATR signaling other than caffeine may also be used. In preferred embodiments, a composition, e.g., a cosmetic composition, is administered which contains caffeine (or other xanthine) at a concentration of less than 14%, preferably less than 12%, more preferably less than 10%, 8%, 7%, 6%, 5%, 4%, 3%, 2%, 1.5%, 1%, 0.5%, or ranges therebetween (including e.g., between 7.5%-12%, 7.5%-10%, 0.5%-7%, 0.01%-0.5%, or 10%-14%). In some embodiments, a cosmetic composition is administered which contains caffeine at a concentration between 0.05-0.5%, 0.5-8%, preferably between 0.5-6%, more preferably between 0.5-5% caffeine. Other agents that decrease ATR signalling include inhibitors of ATR, such as pentoxyfylline. The composition may include or excludes a UV filter. The composition may include a sapogenin, or may include a sapogenin, e.g., may be substantially free of a sapogenin.

The method can be used prophylactically (e.g., before wrinkles are apparent) or they can be used to prevent further wrinkle formation or reduce the appearance of wrinkles in a subject.

In another aspect, the disclosure features a method for identifying an agent that modulates, e.g., reduces, UVB-induced wrinkles. The method includes identifying an agent that modulates, e.g., decreases, ATR and/or ATR-mediated signaling (e.g., an agent that the reduces the expression, activity or levels of ATR or of an ATR binding partner (e.g., ATRIP (ATR-interacting protein), or a downstream ATR effector, such as CHK1), or UV damaged DNA. In some cases, the agent modulates ATR interactions with UV-damaged DNA, for example, it inhibits such interactions, e.g., by reducing affinity of ATR for UV-damaged DNA. For example, the agent reduces such activity (or levels) by at least about 25, 50, 75, 80, or 90%.

The method can include correlating decreased expression, activity or levels of a component of the ATR-mediated replication checkpoint cascade with the agent's ability to prevent or reduce wrinkles, e.g., identifying the agent as a wrinkle protection or reduction agent (e.g., providing print material or a computer readable medium, e.g., informational, marketing or instructional print material or computer readable medium, related to the identified agent or its use). Correlating can include identifying a test agent that decreases expression, activity or levels of a component of the ATR-mediated replication checkpoint cascade (e.g., a test agent that decreases ATR expression, levels or activity) as an agent capable of preventing, reducing or treating wrinkles. The correlating step can include generating or providing a record, e.g., a print or computer readable record, such as a laboratory record or dataset or an email, identifying a test agent that decreases expression, activity or levels of a component of the ATR-mediated replication checkpoint cascade as an agent capable of preventing, reducing or treating wrinkles. In one embodiment, the method includes correlating a value for the effect of the agent with ability to reduce skin damage, e.g., generating a dataset correlating a value for the effect of the agent with ability to reduce skin damage. The value may be preferably statistically significant, e.g., using the Student's T test. The record or dataset can include other information, such as a specific test agent identifier, a date, an operator of the method, or information about the source, structure, method of purification or biological activity of the test agent. The record or information derived from the record can be used, e.g., to identify the test agent as a compound or candidate agent (e.g., a lead compound) for pharmaceutical or therapeutic use. The identified agent can be identified as an agent or a potential agent for treatment or reduction or wrinkles. Agents, e.g., compounds, identified by this method can be used, e.g., in the treatment (or development of treatments, e.g., cosmetic treatments) for wrinkles.

In one embodiment, the method includes evaluating, e.g., measuring, the effect of a test agent on skin, e.g., evaluating a parameter correlated with wrinkles, e.g., the presence, extent, or type of wrinkles; and selecting a test agent that prevents or reduces damage to the skin, e.g., prevents or reduces wrinkles in the skin. Preferably, evaluating the effect of the test agent on skin includes administering the test agent, e.g., topically, to a tissue or subject and comparing a parameter correlated with wrinkles, e.g., the presence, extent, or type of wrinkles in the tissue or subject with a reference value, e.g., a control or baseline value, e.g., a value for the same parameter in a tissue or subject that has been treated differently, e.g., has not been administered the agent. The effect of the agent on skin can be evaluated in the absence or presence of a source of skin damage, e.g., an agent or treatment that induces wrinkle formation, e.g., UVB radiation. In some embodiments, the evaluation includes entering a value for the evaluation, e.g., a value for the presence, extent, or type of wrinkles into a database or other record.

In one embodiment, the agent is identified by evaluating the ability of a test agent to interact with, e.g., to bind, ATR. In another embodiment, the agent is identified by evaluating the effect of a test agent to interact with an ATR regulatory region, e.g., a promoter. In another embodiment, the agent is identified by evaluating the effect of the test agent on ATR production in a skin cell, e.g., in a keratinocyte. In another embodiment, the agent is identified by evaluating, e.g., quantitatively or qualitatively evaluating, the ability of a test agent to modulate ATR signaling in a whole animal model, e.g., in the skin of an ATR transgenic animal such as an ATR overexpressing animal.

The test agent is not limited and can be, e.g., a nucleic acid (e.g., an antisense, ribozyme), a polypeptide (e.g., an antibody or antigen-binding fragment thereof), a peptide fragment, a peptidomimetic, or a small molecule (e.g., a small organic molecule with a molecular weight of less than 2000 Daltons). In another preferred embodiment, the test agent is a member of a combinatorial library, e.g., a peptide or organic combinatorial library, or a natural product library. In a preferred embodiment, a plurality of test agents, e.g., library members, is tested. Preferably, the test agents of the plurality, e.g., library, share structural or functional characteristics. The test agent can also be a crude or semi-purified extract, e.g., a botanical extract such as a plant extract, or algal extract.

In one embodiment, the method includes two evaluating steps, e.g., the method includes a first step of evaluating the test agent in a first system, e.g., a cell-free, cell-based, tissue system or animal model, and a second step of evaluating the test agent in a second system, e.g., a second cell or tissue system or in a non-human animal. A cell-based system can include a ATR-expressing cell, e.g. a yeast, mammal, rodent, or human cell that expresses ATR or an ATR-like protein. For example, the cell can be a non-human cell that is engineered to express human ATR or a functional fragment thereof. In one embodiment, one of the evaluating steps includes evaluating the effect of the agent on a subject's skin or skin explant, e.g., evaluating the presence, extent or type of wrinkles in the skin, preferably before and after UVB exposure. The subject can be an experimental animal or a human. In one embodiment, the first evaluation includes testing the effect of the test agent on an ATR promoter that is linked to a heterologous sequence such as a reporter gene, and the second evaluation includes administering the test agent to a system, e.g., a cell based or animal system and evaluating effect of the agent on skin damage and/or ATR production. In some embodiments, the method includes two evaluating steps in the same type of system, e.g., the agent is re-evaluated in a non-human animal after a first evaluation in the same or a different non-human animal. The two evaluations can be separated by any length of time, e.g., days, weeks, months or years.

In a preferred embodiment, the identifying step includes: (a) providing an agent to a cell, tissue or non-human animal whose genome includes an exogenous nucleic acid that includes a regulatory region (e.g., a promoter) of an ATR gene (see, e.g., GenBank LocusID No. 545; GenBank Identifier NM_{—}001184, and the chromosome III contig NT_{—}005612 of build 34 version 3 of the NCBI's genome annotation; the regulatory region can include, e.g., a region that is within 500 or 1000 basepairs of nucleotide 48663233 or 48792805 of the NT_{—}005612 contig, e.g., a region upstream or downstream of complement(48663233.48792805)), operably linked to a heterologous sequence, e.g., a nucleotide sequence encoding a reporter polypeptide (e.g., a colorimetric (e.g., LacZ), luminometric, e.g., luciferase, or fluorescently detectable reporter polypeptide, e.g. GFP, EGFP, BFP, RFP); (b) evaluating the ability of a test agent to modulate the expression of the reporter polypeptide in the cell, tissue or non-human animal; and (c) selecting a test agent that modulates (e.g., reduces) the expression of the reporter polypeptide as an agent that modulates (e.g., reduces) UVB-induced wrinkles.

In one embodiment, the animal is an experimental rodent. The animal can be wild type or a transgenic experimental animal, e.g., an ATR transgenic rodent, e.g., an ATR transgenic mouse described herein. The subject can also be a non-human mammal or a human.

In a preferred embodiment, the evaluating step comprises administering the agent to the subject and evaluating skin damage (e.g., skin damage caused by acute exposure to UVB). In another embodiment, the cell or tissue is a skin cell, e.g., a keratinocyte; or tissue, e.g., a skin explant. In yet another embodiment, a cell, e.g., a skin cell, e.g., a keratinocyte, or a tissue, e.g., a skin explant, is derived from a transgenic animal.

In another aspect, the disclosure features compositions containing an agent, e.g., an agent described herein, e.g., an agent identified by a screening method described herein, that decreases the expression, activity, or level of ATR, for reducing UVB-induced wrinkles. In a preferred embodiment, the composition is a cosmetic composition, e.g., formulated for topical administration. In a preferred embodiment, the composition also has a fragrance, a preservative, or other cosmetic ingredient, e.g., a moisturizer, or sunscreen agent, e.g., octyl methoxycinnamate, aminobenzoic acid, oxybenzone, padimate O, homosalate, or titanium dioxide. The composition can be provided in a shampoo, oil, cream, lotion, soap, foam, gel, or other cosmetic preparation. In a preferred embodiment, the composition also has a cosmetic ingredient, e.g., a fragrance or moisturizer. The composition may include other active agents, e.g., biologically active agents including, e.g., a retinol.

In another aspect, the disclosure features a method of modulating skin damage in a subject. The method includes supplying to the subject a composition containing an agent that affects the expression, activity or level of a component of ATR signaling, e.g., an agent described herein, e.g., an agent identified by a screening method described herein, and supplying to the subject application instructions for acute UVB-induced wrinkles.

In another aspect, the disclosure features a kit for modulating skin damage of a subject that includes a composition described herein, e.g., a composition containing an agent that affects the expression, activity, or level of a component of ATR signaling; and instructions for use, e.g., instructions to apply the composition to an area of the body in need of treatment for UVB-induced skin damage, e.g., wrinkles. In a preferred embodiment, the composition also has a cosmetic ingredient, e.g., a fragrance or moisturizer.

In another aspect, the disclosure features a method of modulating angiogenesis in the skin of a subject. The method includes applying an agent described herein to a site at which modulation of angiogenesis is required. The method can include evaluating skin of a subject to identify or characterize a site, as a site at which modulation of angiogenesis is required. For example, at a site of a melanoma or potential melanoma, it can be useful to reduce angiogenesis.

In another aspect, the disclosure features a method for providing a composition (e.g., a cosmetic composition). The composition can be a composition for treating wrinkles and can include an agent that affects the expression, activity, or level of a component of ATR signaling. The method can include (e.g., as part of a production or quality control method) sampling a preparation of the composition and evaluating the sample for ability to modulate (e.g., inhibit) expression, activity, or level of a component of ATR signaling, e.g., using an in vitro or cell based assay.

An “effective amount” of the agent is the amount of a composition that, upon administration to a subject, reduces UV-induced wrinkles in the subject. The effective amount to be administered to a subject is typically based on a variety of factors including age, sex, surface area, weight, and conditions of the skin. Body surface area may be approximately determined from height and weight of the patient. See, e.g., Scientific Tables, Geigy Pharmaceuticals, Ardley, N.Y., 1970, 537. Effective doses will vary, as recognized by those skilled in the art, dependent on route of administration, excipient usage, and the possibility of co-usage with other treatments such as usage of other skin damage-modulating compounds.

As used herein, an “agent that modulates ATR signalling” is an agent that causes at least a 10% change in ATR signalling activity when present in cell culture at millimolar concentration or less using the chromosomal fragile site assay as described in Casper, Nghiem, et al (2002) Cell 111(6):779-89, or the premature chromatin condensation assay as described in Nghiem, et al (2001) Proc Natl Acad Sci USA. 98(16):9092-7.

The details of one or more embodiments of the invention are set forth in the accompanying drawings and the description below. Other features, objects, and advantages of the inventions will be apparent from the description and drawings, and from the claims.

### DETAILED DESCRIPTION

ATR (ATM-Rad3-related) is a protein kinase of the phosphoinositide 3-kinase-related kinase (PIK) gene superfamily. ATR functions as proximal DNA damage-signaling kinase that is involved in cell cycle checkpoint activation. In particular, ATR is required for the DNA replication checkpoint, which delays mitosis in the presence of unreplicated DNA. ATR is also required to prevent replication fork collapse and DNA strand breakage when DNA replication is transiently inhibited. Consistent with its critical S phase functions, ATR is an essential gene.

ATR can be activated by a variety of DNA damaging agents, e.g., UV exposure. ssDNA lesions created during the repair of UV damage are not sufficient to activate the ATR-dependent pathway. ATR activation is only observed in replicating cells indicating that replication stress is required to trigger the ATR-mediated checkpoint cascade in response to UV irradiation (Ward et al., 2004, J. Biol. Chem., 279:9677-9680)

ATR has been implicated as a tumor suppressor and has been found to be mutated in certain human cancers. Caffeine can inhibit ATR activation.

Exposure to UVB Radiation

The major source of UVB radiation is natural sunlight. The intensity of UVB rays varies depending on the time of day, time of year, the sun's position in the sky, altitude and distance from the equator. These rays are most intense during the midday hours in the summer, although they are always present, even during the winter months. Distance above sea level and distance from the equator are also important to consider. The higher the altitude the greater the intensity of UVB rays. Therefore, mountaineers, skiers, and those who live at high altitudes are at risk of long term UVB damage. Also, the nearer one is to the equator the more intense the UV radiation and the higher the risk of long term UVB damage.

Snow, water, and sand reflect sunlight, magnifying the amount of UVB radiation that reaches the skin. Even when clouds obscure the sun, UVB levels can still be sufficiently high to cause photoaging, e.g., wrinkles, upon long term exposure.

The UV index (developed by the Environmental Protection Agency) indicates the intensity of the sun's UV rays on a given day. There are four categories—moderate (UV index is less than 3), high (UV index is 3 to 6) very high (UV index is 6 to 10) and extreme (UV index is greater than 10). A moderate UV Index means it will take more than an hour to burn your skin; an extreme level means it will take less than 15 minutes. The index is often included with weather reports. Clinically, UVB exposure is measured in MEDs. One MED is the amount of UVB required to produce a sunburn in sensitive skin. Because the effects of UVB exposure are cumulative, long term or chronic UVB induced wrinkles can occur as a result of long term exposure to UVB levels below those which, upon acute exposure, can cause erythema or edema or burning (e.g., below one MED, below 0.5 MED, or less). For example, a subject is at risk of long term UVB-induced wrinkles if the subject is chronically exposed to the sun even if the subject is only exposed to the sun during days with a low or moderate UV Index.

Wrinkles

Wrinkles can result from numerous causes. Wrinkles can be caused, inter alia, from the natural aging process of the skin, from smoking, and from exposure to the ultraviolet radiation (e.g., from chronic sun exposure). A wrinkle is a configuration change in the surface of the skin, without specific structural alterations at the histological level. Generally, wrinkles are classified as described in Kligman et al. (1985) Br J Derm 113:37-42, herein incorporated by reference. Kligman classifies wrinkles into three classes: linear wrinkles, glyphic wrinkles, and crinkles. Linear wrinkles are straight, found generally in the facial skin, and can be caused. by natural aging or exposure to ultraviolet light. Glyphic wrinkles are shaped as apparent triangles or rectangles of wrinkles, are found on the face, hands, and neck exposed to sunlight, and are aggravated by exposure to ultraviolet light or dermatoheliosis. Crinkles are thin, crinkled wrinkles on flabby skin, found anywhere on the skin, but typically on the backs of hands and around the eyelids.

Linear wrinkles can be further subclassified into (a) regular wrinkles and (b) fine wrinkles. Regular wrinkles are long, deep, clear, and are also referred to as crow's feet. Fine wrinkles are thin and shallow. Regular wrinkles have a width of at least about 155 microns (0-32 Hz), preferably about 160 to 250 microns. Fine wrinkles have a width of less than about 154 microns, preferably about 40 to 154 microns (32-126 Hz), as calculated e.g., in a power spectrum obtained through transforming three dimensional shape data into data in a frequency domain by two-dimensional Fourier transformation (using, e.g., the Shiseido Wrinkle Analyzer 3D Pro system, essentially as described in Takasu et al. (1996) J Soc Cosmet Chem Japan 29:394-405; and Japanese Published Patent Application No. 07-113623, published May 2, 1995).

The methods herein disclosed to prevent or treat or reduce UV-induced wrinkles in a subject include administering to the subject an agent that inhibits ATR and/or ATR mediated checkpoint signaling. An exemplary treatment method can include locating a wrinkle or a potential site of wrinkling and applying a composition described herein.

Screening Methods

Numerous methods exist for evaluating whether an agent can modulate ATR signaling, e.g., ATR gene expression, activity or level. In one embodiment, the ability of a test agent to modulate, e.g., decrease, e.g., permanently or temporarily, expression from the ATR gene promoter is evaluated by routine reporter transcription assay (e.g., LacZ or GFP or luciferase). For example, a cell or transgenic animal whose genome comprises a reporter gene operably linked to an ATR promoter, can be contacted with a test agent, and the ability of the test agent to increase or decrease reporter activity is indicative of the ability of the agent to modulate UVB induced wrinkles. In another embodiment, the ability of a test agent to modulate ATR gene expression, or ATR activity or level, is evaluated in a transgenic animal, for example, the transgenic animal described herein.

The effect of a test agent on ATR gene expression or ATR activity or level may also be evaluated in a cell, cell lysate, or subject, preferably a non-human experimental mammal, and more preferably a rodent (e.g., a rat, mouse, or rabbit), or explant (e.g., skin) thereof. Methods of assessing ATR gene expression are well know in the art, e.g., Northern analysis, ribonuclease protection assay, reverse transcription-polymerase chain reaction (RT-PCR) or RNA in situ hybridization (see, e.g., Sambrook et al. Molecular Cloning: A Laboratory Manual (3^{rd} ed. 2001)). The level of ATR may be monitored by, e.g., Western analysis, immunoassay, or in situ hybridization. ATR activity, e.g., altered promoter binding and/or transcription activity, may be determined by, e.g., electrophoretic mobility shift assay, DNA footprinting or reporter gene assay. Preferably, the effect of a test agent on ATR gene expression or ATR activity or level is observed as a change in skin damage in a subject. More preferably, the effect of a test agent on ATR gene expression or ATR activity or level is evaluated on a transgenic cell or non-human animal, or explant or cell derived therefrom, having altered ATR signaling, as compared to a wild-type cell or non-human animal, or explant or cell derived therefrom.

The test agent may be administered to a cell, cell extract, explant or subject expressing a transgene comprising the ATR gene promoter fused to LacZ. (Enhancement or inhibition of transgene, e.g., a reporter, e.g., LacZ or GFP, transcription, as a result of an effect of the test agent on the ATR gene promoter or factors regulating transcription from the ATR gene promoter, may be easily observed as a change in color. Reporter transcript levels, and thus ATR gene promoter activity, may be monitored by established methods, e.g., Northern analysis, ribonuclease protection assay, reverse transcription-polymerase chain reaction (RT-PCR) or RNA in situ hybridization (see, e.g., Cuncliffe et al. (2002) Mamm. Genome 13:245). Agents may be evaluated using a cell-free system, e.g., an environment comprising the ATR gene promoter-reporter transgene (e.g., ATR gene promoter-LacZ transgene), transcription factors binding the ATR gene promoter, a crude cell lysate or nuclear extract, and the test agent (e.g., an agent described herein), wherein an effect of the agent on ATR gene promoter activity is detected as a color change.

ATR protein or fragment thereof for use in screening assays can be produced, e.g., using a recombinant nucleic acid encoding the protein or corresponding fragment. Cimprich et al. (1996) Proc Natl Acad Sci USA. 1996 Apr. 2;93(7):2850-5 describe an exemplary ATR mRNA sequence which can be used to produce an expression construct. Exemplary fragments include, e.g., amino acids about 1640-2185, 2612-2644, 2321-2567, 2321-2633, the HEAT domain, the FAT domain, the FATC domain, the PI-3/PI-4 kinase domain, or the TPR-like domain of ATR. Such fragments or the full length protein can be produced in recombinant cells and purified, or can be evaluated in the cell, e.g., as in a two-hybrid assay. Sambrook & Russell, Molecular Cloning: A Laboratory Manual, 3^{rd} Edition, Cold Spring Harbor Laboratory, N.Y. (2001) and Ausubel et al., Current Protocols in Molecular Biology (Greene Publishing Associates and Wiley Interscience, N.Y. (1989), for example, provide general cloning and recombinant protein expression methods. Scopes (1994) Protein Purification: Principles and Practice, New York: Springer-Verlag, for example, and other texts provide a number of general methods for purifying recombinant (and non-recombinant) proteins. Ünsal-Kacmaz et al. (2002) Proc Natl Acad Sci USA. 2002 May 14; 99 (10): 6673-6678 also describes purifying full length flag-tagged ATR protein.

It is possible to evaluate one or more ATR activities in vitro. For example, Ünsal-Kacmaz et al. (2002) Proc Natl Acad Sci USA. 2002 May 14; 99 (10): 6673-6678 describes exemplary DNA binding assay in which interaction between purified ATR protein and UV damaged DNA is evaluated and a ATR kinase assay. Candidate compounds may decrease DNA binding by at least 25, 50, 75, 80, or 90% or decrease kinase activity at least 25, 50, 75, 80, or 90%.

It is also possible to evaluate ATR activity in cells, e.g., in tissue culture cells. Exemplary cellular assays are described in Nghiem et al. (2002) J Biol. Chem. 2002 Feb 8;277(6):4428-34 and Nghiem et al. (2001) Proc Natl Acad Sci USA. 2001 Jul 31;98(16):9092-7.

Pharmacokinetic Properties and Therapeutic Activity

Modifications can be made to an agent described herein that result in pharmacokinetic properties of the agent which are desirable for use in therapy. For example, such modifications can result in longer circulatory half-life, an increase in cellular uptake, improved distribution to targeted tissues, a decrease in clearance and/or a decrease of immunogenicity. Several art-recognized approaches useful to optimize the therapeutic activity of an agent, e.g., a xanthine described herein, e.g., caffeine. Methods for producing and purifying caffeine and other xanthines are well known.

Expression System

In cases where the protein is an agent, it can be produced as a recombinant protein. For recombinant proteins, the choice of expression system can influence pharmacokinetic characteristics. Differences between expression systems in post-translational processing lead to recombinant proteins of varying molecular size and charge, which can affect circulatory half-life, rate of clearance and immunogenicity, for example. The pharmacokinetic properties of the protein may be optimized by the appropriate selection of an expression system, such as selection of a bacterial, viral, or mammalian expression system. Exemplary mammalian cell lines useful in expression systems for therapeutic proteins are Chinese hamster ovary, (CHO) cells, the monkey COS-1 cell line and the CV-1 cell line.

Chemical Modification

An agent can be chemically altered to enhance the pharmacokinetic properties while maintaining activity. The agent can be covalently linked to a variety of moieties, altering the molecular size and charge of the agent and consequently its pharmacokinetic characteristics. The moieties are preferably non-toxic and biocompatible. In one embodiment, poly-ethylene glycol (PEG) can be covalently attached to a protein (PEGylation). PEG is a class of polymers comprised of repeating ethylene oxide subunits with terminal hydroxyl groups. A variety of PEG molecules are known and/or commercially available (See, e.g., Sigma-Aldrich catalog). Another exemplary modification is the conjugation of arginine oligomers to the agent to facilitate topical delivery (Rothbard et al., 2000, Nat Med. 6(11):1253-7).

Furthermore, a therapeutic agent may be chemically linked to a protein. The therapeutic agent can be cross-linked to a carrier protein to form a larger molecular weight complex with longer circulatory half-life and improved cellular uptake. In one embodiment, the carrier protein can be a serum protein, such as albumin. The therapeutic agent can be attached to one or more albumin molecules via a bifunctional cross-linking reagent. The cross-linking reagent may be homo- or heterofunctional.

Modification of Formulation

The formulation of the agent (e.g., caffeine or other ATR modulating xanthine) may be adapted depending on the desired mode of administration. For example, a therapeutic agent can be formulated in a carrier system.

The carrier can be a colloidal system. The colloidal system can be liposome, a phospholipid bilayer vehicle. In one embodiment, a therapeutic agent is encapsulated in a liposome. As one skilled in the art would appreciate, there are a variety of methods to prepare liposomes. (See Lichtenberg, D., et al., Methods Biochem Anal, 33:337-462 (1988), LIPOSOME TECHNOLOGY Anselem, S. et al., CRC Press, 1993). Liposomes can be prepared from an assortment of phospholipids varying in size and substitution, and may also contain additional components with low toxicity, such as cholesterol. The liposome can be formulated and isolated in a variety of shapes and sizes. Additionally, moieties may attached to the surface of the liposome to further enhance the pharmacokinetic properties of the carrier. The moieties may be attached to phospholipid or cholesterol molecules, and the percentage of the moiety incorporated on the surface may be adjusted for optimal liposome stability and pharmacokinetic characteristics. One embodiment comprises a liposome with poly-ethylene glycol (PEG) added to the surface. Liposomal formulations can delay clearance and increase cellular uptake. (See Reddy, K. R., Annals of Pharmacotherapy, 34:7/8, 915-923 (2000)).

The carrier can also be a polymer, e.g., a biodegradable, biocompatible polymer matrix. In one embodiment, the therapeutic agent can be embedded in the polymer matrix while maintaining protein integrity. The polymer may be natural, such as polypeptides, proteins or polysaccharides, or synthetic, such as poly(α-hydroxy) acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, K. R., Annals of Pharmacotherapy, 34:7/8, 915-923 (2000)). A polymer formulation for human growth hormone (hGH) has been used in clinical trials. (See Kozarich, J. W., Rich, D. H., Chemical Biology 2:548-552 (1998)).

Examples of polymer microsphere sustained release formulations are described in PCT publication WO 99/15154 (Tracy et al.), U.S. Pat. Nos. 5,674,534 and 5,716,644 (both to Zale et al.), PCT publication WO 96/40073 (Zale et al.), and PCT publication WO 00/38651 (Shah et al.). U.S. Pat. Nos. 5,674,534 and 5,716,644 and PCT publication WO 96/40073 describe a polymeric matrix containing particles of erythropoietin that are stabilized against aggregation with a salt.

In one embodiment, the composition has a viscosity of not more than about 15,000 cP, preferably between about 100 and about 12,000, and more preferably between about 300 and about 10,000. A polymeric material can be added to the composition to achieve the desired viscosity. The viscosity is determined at room temperature (20-25° C.) using a Brookfield viscometer model DV-I+, spindle #27 at 12 revolutions per minute (rpm). If the measured viscosity is less than 4,000 cP, spindle #21 should be used instead of #27. By keeping the viscosity below about 15,000 cP, the advantages of appealing cosmetic characteristics and ease of accurate application through improved flow and pourability are achieved.

The polymers that can be particularly useful are lightly cross-linked polyacrylic acid polymers, e.g., such as are available from B. F. Goodrich under the tradename CARBOPOL™. They are generically referred to as carbomers. The CARBOPOL™ polymers are hydrophilic polymers based on a polyacrylic acid structure. Examples of lightly cross-linked polymers include CARBOPOL™ 910, 941,971, and 981 and CARBOPOL™ ETD 2050. Either CARBOPOL™ 941 or 981 is useful because the viscosity of a gel based on CARBOPOL™ 941 or 981 is low relative to its concentration due to the low level of cross-linking within the polymer structure in a neutralized aqueous system. In contrast polyacrylic acid polymers which display a high level of cross-linking, such as CARBOPOL™ 980 or 974P, produce gels with higher viscosity at comparable concentrations.

A 0.5% solution of either CARBOPOL™ 941 or 981 at pH 7.5 has a viscosity measurement of from 4,000 to 11,000 cP (Brookfield viscometer at 20 rpm) compared to a viscosity measurement of from 40,000 to 60,000 cP for a comparable 0.5% solution of either CARBOPOL™ 940 or 980 (reference: B. F. Goodrich Product Guide, Bulletin 2). A gel made from one of these lightly cross-linked polymers provides better skin feel and lubricity than a gel of comparable viscosity made from a highly cross-linked polymer. A low viscosity gel can also be administered very accurately by a dropper or drip-type dispenser as compared to other commercial products which are thicker gels that do not provide as accurate an application.

CARBOPOL™ 941 NF resin and its cosolvent polymerized alternative, CARBOPOL™ 981 NF resin, provide permanent emulsions and suspensions at low viscosities. The gels produced with these resins have excellent clarity. In ionic systems, they perform better than most of the other CARBOPOL™ resins and at concentrations below 1.5% in solvent systems. The polymers are available from B. F. Goodrich Specialty Chemicals, 9911 Brecksville Road, Cleveland, Ohio 44414-3247. CARBOPOL™ resins are polymers of acrylic acid crosslinked with polyalkenyl ethers or divinyl glycol.

A composition may also include a preservative, e.g., an component that aids in ensuring a stable composition and/or prevents growth of bacteria. The preservative may be one or more of an antioxidant, a chelator, an antibacterial, or the like. Suitable preservatives include methylparaben, butylparaben, propylparaben, benzyl alcohol, sorbic acid, imidurea, thimerisal, propyl gallate, BHA, BHT, citric acid, disodium edetate, and the like. Another optional additive is a fragrance. Generally, this will be present in a trace amount only and has no effect on the functioning of the composition.

Antisense Nucleic Acid Sequences

Nucleic acid molecules that are antisense to a nucleotide encoding a component of the ATR-mediated replication checkpoint cascade, e.g., ATR, can be used as an agent that prevents or reduces UVB-induced wrinkles in the methods and compositions described herein. An “antisense” nucleic acid includes a nucleotide sequence which is complementary to a “sense” nucleic acid encoding a component of the ATR-mediated replication checkpoint cascade, e.g., ATR, e.g., complementary to the coding strand of a double-stranded cDNA molecule or complementary to an mRNA sequence. Accordingly, an antisense nucleic acid can form hydrogen bonds with a sense nucleic acid. The antisense nucleic acid can be complementary to an entire coding strand, or to only a portion thereof. For example, an antisense nucleic acid molecule which antisense to the “coding region” of the coding strand of a nucleotide sequence encoding ATR can be used.

Given the coding strand sequence encoding ATR or any other component of the ATR-mediated replication checkpoint cascade, antisense nucleic acids can be designed according to the rules of Watson and Crick base pairing. The antisense nucleic acid molecule can be an oligonucleotide which is antisense to only a portion of the coding or noncoding region of ATR mRNA. For example, the antisense oligonucleotide can be complementary to the region surrounding the translation start site of ATR mRNA. An antisense oligonucleotide can be, for example, about 5, 10, 15, 20, 25, 30, 35, 40, 45 or 50 nucleotides in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest.

RNAi

Double stranded nucleic acid molecules that can silence a gene can also be used as an agent to inhibit expression of a component of the ATR-mediated replication checkpoint cascade, e.g., ATR. RNA interference (RNAi) is a mechanism of post-transcriptional gene silencing in which double-stranded RNA (dsRNA) corresponding to a gene (or coding region) of interest is introduced into a cell or an organism, resulting in degradation of the corresponding mRNA. The RNAi effect persists for multiple cell divisions before gene expression is regained. RNAi is therefore an extremely powerful method for making targeted knockouts or “knockdowns” at the RNA level. RNAi has proven successful in human cells, including human embryonic kidney and HeLa cells (see, e.g., Elbashir et al. Nature 2001 May 24;411(6836):494-8). In one embodiment, gene silencing can be induced in mammalian cells by enforcing endogenous expression of RNA hairpins (see Paddison et al.,2002, PNAS USA 99:1443-1448). In another embodiment, transfection of small (21-23 nt) dsRNA specifically inhibits gene expression (reviewed in Caplen (2002) Trends in Biotechnology 20:49-51). Such small dsRNAs can include RNAs referred to as siRNAs or short interfering RNAs.

dsRNA corresponding to a portion of a gene to be silenced can be introduced into a cell. The dsRNA is digested into 21-23 nucleotide siRNAs, or short interfering RNAs. The siRNA duplexes bind to a nuclease complex to form what is known as the RNA-induced silencing complex, or RISC. The RISC targets the homologous transcript by base pairing interactions between one of the siRNA strands and the endogenous mRNA. It then cleaves the mRNA˜12 nucleotides from the 3′ terminus of the siRNA (reviewed in Sharp et al (2001) Genes Dev 15: 485-490; and Hammond et al. (2001) Nature Rev Gen 2: 110-119).

RNAi technology in gene silencing utilizes standard molecular biology methods. dsRNA corresponding to the sequence from a target gene to be inactivated can be produced by standard methods, e.g., by simultaneous transcription of both strands of a template DNA (corresponding to the target sequence) with T7 RNA polymerase. Kits for production of dsRNA for use in RNAi are available commercially, e.g., from New England Biolabs, Inc. Methods of transfection of dsRNA or plasmids engineered to make dsRNA are routine in the art.

Gene silencing effects similar to those of RNAi have been reported in mammalian cells with transfection of a mRNA-cDNA hybrid construct (Lin et al., Biochem Biophys Res Commun 2001 Mar. 2;281(3):639-44), providing yet another strategy for gene silencing. Agents that can be used to decrease ATR signaling include RNAi's that reduce ATR expression, e.g., siRNA or larger dsRNAs that include a sequence complementary to a ATR mRNA, e.g., the coding region thereof, the 5′ or 3′ half of the coding region thereof. Cimprich et al. (1996) Proc Natl Acad Sci USA. 1996 Apr. 2;93(7):2850-5 describe an exemplary ATR mRNA sequence. In addition, other nucleic acid agents such as anti-sense RNAs, ribozymes, and PNA's can also be used.

Accordingly, RNAi (such as dsRNAs and siRNAs) that reduce expression of a component of the ATR-mediated replication checkpoint cascade, e.g., ATR can be used. For example, such RNAs may include a region that is complementary to a gene encoding such a component, e.g., a gene encoding ATR.

Antibodies

Antibodies that bind (and preferably inhibit) a component of the ATR-mediated replication checkpoint cascade, e.g., ATR, can be used in the methods and compositions described herein. Methods for making monospecific antibodies and antibody fragments are known in the art and can be found, e.g., in Zola, Monoclonal Antibodies: Preparation and Use of Monoclonal Antibodies and Engineered Antibody Derivatives. Springer Verlag (Dec. 15, 2000; 1st edition).

Monospecific antibodies are not limited to monoclonal antibodies produced by a hybridoma, but also include monospecific antibodies that have been artificially modified, e.g., for the purpose of lowering heteroantigenicity to humans. Examples include chimeric, reshaped and humanized antibodies. For example, a chimeric antibody can be made that is composed of the variable regions of the monoclonal antibody of a mouse or other non-human mammal and constant regions of human antibody. This type of chimeric antibody can be produced using known methods for producing chimeric antibodies and particularly gene recombination technology. A reshaped antibody is one in which the complementarity determining regions (CDR) of a human antibody are replaced with the complementarity determining regions of an antibody of a non-human mammal such as a mouse, and its general gene recombination techniques are known. A reshaped human antibody can be obtained by using these known methods. Furthermore, amino acids of the framework (FR) regions of the variable region of antibody may be substituted so as to form a suitable antigen binding site in the complementarity determining regions of the reshaped human antibody (Sato et al., Cancer Res. 53:1-6, 1933). The making of such a reshaped human antibody is exemplified in International Patent Application No. WO92-19759.

Moreover, a gene can be constructed that codes for antibody fragments, such as Fab or Fv, or a single chain Fv (scFv) in which Fv of the H chain and L chain are connected with a suitable linker. This gene can be expressed in a suitable host cell and used for the purpose described above, provided it binds to antigen and inhibits the activity of antigen (see, for example, Bird et al., TIBTECH 9:132-137, 1991; Huston et al., Proc. Natl. Acad. Sci. USA 85: 5879-5883, 1988). Moreover, the V region of the above-mentioned reshaped antibody can be used for the Fv of the H chain and L chain used for producing scFv.

In addition, a monospecific antibody can be a human antibody. The human antibody can be obtained, e.g., by isolating cells producing the human antibody or cloning the human antibody gene isolated from the cells producing the human antibody. For example, a transgenic animal (e.g., a mouse) in which the original immune system has been replaced with the human immune system, can be immunized to produce a fully human antibody. In addition to this, the technologies of immortalizing and cloning human peripheral blood lymphocytes are known in the art. Human antibodies are described, e.g., in Sanz et al., 2004, Trends Immunol. 25(2):85-91.

Administration

An agent described herein may be administered systemically or locally, e.g., topically. Topical administration of an agent described herein is the preferred route of administration. Topical compositions that include the agent can exist in many forms, e.g., in the form of a solution, cream, ointment, gel, lotion, shampoo, soap or aerosol. A wide variety of carrier materials can be employed, such as alcohols, aloe vera gel, allantoin, glycerin, vitamin A and E oils, mineral oils, and polyethylene glycols. Other additives, e.g., preservatives, fragrance, sunscreen, or other cosmetic ingredients, can be present in the composition. The composition is typically not in the form of an aqueous liquid. Examples of preservatives include phenoxyethanol and parabens such as methyl-paraben, ethyl-paraben, and propyl-paraben; salicylic acid, chlorhexidine hydrochloride, phenoxyethanol, sodium benzoate, methyl para-hydroxybenzoate, ethyl para-hydroxybenzoate, propyl para-hydroxybenzoate, butyl para-hydroxybenzoate, isothiazolones and the like.

The agent can also be administered using a topical applicator. For example, the composition that includes the agent can be a component of a band-aid, tape, bandage, article of clothing, patch, and so forth. The composition may be delivered by a variety of methods including direct contact and aerosolized delivery. For example, the composition can be atomized and sprayed onto a surface, e.g., skin at a desired location. The composition may also be delivered by iontophoresis.

One preferred vehicle for topical delivery is liposomes. Liposomes can be used to carry and deliver an agent, e.g., an agent described herein, into a cell. Detailed guidance can be found in, e.g., Yarosh et al. (2001) Lancet 357: 926 and Bouwstra et al. (2002) Adv. Drug Deliv. Rev. 54 Suppl 1:S41

For systemic administration the agent may be administered via the oral route or the parenteral route, including subcutaneously, intraperitoneally, intramuscularly, intravenously or other route. For local administration, they are administered topically, transdermally, transmucosally, intranasally or other route. A cell may be contacted extracellularly or intracellularly with the agent, e.g., by microinjection or transfection. The agent may be applied and removed immediately, applied and not removed, and/or repeatedly applied with constant, increasing or decreasing frequency and/or at increasing or decreasing doses or concentrations. More than one route of administration may be used simultaneously, e.g., topical administration in association with oral administration. Examples of parenteral dosage forms include aqueous solutions of the active agent, in a isotonic saline, 5% glucose or other well-known pharmaceutically acceptable excipient. Solubilizing agents such as cyclodextrins, or other solubilizing agents well known to those familiar with the art, can be utilized as pharmaceutical excipients for delivery of the pigment modulating composition.

The composition may be provided as, e.g., a cosmetics, a medication or a skin care product. The composition can also be formulated into dosage forms for other routes of administration utilizing conventional methods. A pharmaceutical composition can be formulated, for example, in dosage forms for oral administration as a powder or granule, or in a capsule, a tablet (each including timed release and sustained release formulations), or a gel seal, with optional pharmaceutical carriers suitable for preparing solid compositions, such as vehicles (e.g., starch, glucose, fruit sugar, sucrose, gelatin and the like), lubricants (e.g., magnesium stearate), disintegrators (e.g., starch and crystalline cellulose), and binders (e.g., lactose, mannitol, starch and gum arabic). When the composition is an injection, for example, solvents (e.g., distilled water for injection), stabilizers (e.g., sodium edetate), isotonizing agents (e.g., sodium chloride, glycerin and mannitol), pH-adjusting agents (e.g., hydrochloric acid, citric acid and sodium hydroxide), suspending agents (e.g., methyl cellulose) and the like may be used.

The composition can include caffeine-sodium benzoate. For example the compounds can be in 50:50 (w/w) mixture, e.g., as provided in C-4144 (Sigma Aldrich), other combinations can also be used, e.g., between 10:90 to 40:60 (w/w) or between 40:60 to 60:40 (w/w) or between 60:40 to 90:10 (w/w). The composition can be applied to a subject who does not have a detectable skin cancer, or can be applied (e.g., topically) to a subject in an area that is free of a neoplasia or other cancer, e.g., free of a skin cancer.

The agent may contain other pharmaceutical ingredients, e.g., a second treatment for skin, e.g., a moisturizer, a sunscreen. In certain embodiments, the composition is substantially free of glycerin or has less than 28% glycerin, although in some cases glycerin is used.

Kits

An agent described herein (e.g., an anti-ATR antibody or an agent that modulates ATR) can be provided in a kit. The kit includes (a) the agent, e.g., a composition that includes the agent, and (b) informational material. The informational material can be descriptive, instructional, marketing or other material that relates to the methods described herein and/or the use of the agent for the methods described herein. For example, the informational material relates to UVB-induced skin damage, e.g., wrinkles.

In one embodiment, the informational material can include instructions to administer an agent described herein in a suitable manner to perform the methods described herein, e.g., in a suitable dose, dosage form, or mode of administration (e.g., a dose, dosage form, or mode of administration described herein). Preferred doses, dosage forms, or modes of administration are topical and cosmetic formulations. In another embodiment, the informational material can include instructions to administer an agent described herein to a suitable subject, e.g., a human, e.g., a human having, or at risk for, UVB damage, e.g., wrinkles.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about ATR and/or its use in the methods described herein. Of course, the informational material can also be provided in any combination of formats.

In addition to an agent described herein, the composition of the kit can include other ingredients, such as a solvent or buffer, a stabilizer, a preservative, a fragrance or other cosmetic ingredient, and/or a second agent for treating a condition or disorder described herein. Alternatively, the other ingredients can be included in the kit, but in different compositions or containers than an agent described herein. In such embodiments, the kit can include instructions for admixing an agent described herein and the other ingredients, or for using an agent described herein together with the other ingredients.

An agent described herein can be provided in any form, e.g., liquid, dried or lyophilized form. An agent described herein be substantially pure and/or sterile. When an agent described herein is provided in a liquid solution, the liquid solution preferably is an aqueous solution, with a sterile aqueous solution being preferred. When an agent described herein is provided as a dried form, reconstitution generally is by the addition of a suitable solvent. The solvent, e.g., sterile water or buffer, can optionally be provided in the kit.

The kit can include one or more containers for the composition containing an agent described herein. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of an agent described herein. For example, the kit includes a plurality of syringes, ampules, foil packets, or blister packs, each containing a single unit dose of an agent described herein. The containers of the kits can be air tight and/or waterproof.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a swab.

### EXAMPLES

### Example 1

### Topical Caffeine does not Cause Skin Irritation

The following example was performed to determine if caffeine causes skin irritation. Skin irritants may contribute to promote wrinkle formation.

Prior to the experiment, the thickness of mice ears (FVB strain, female, 7 weeks of age, n=3-4/group) was measured with a thickness gauge (Mitsutoyo Corp.). After measuring the thickness of both ears, 10 μl of solvent was applied on the left ear and 10 μl of caffeine solution (2% or 1.2%) was applied on the right ear. The following day, ear thickness of both ears was measured. This procedure was continued for five consecutive days. The results of both the left and right ear thickness are tabulated in Table 1 below.

TABLE 1

| DAY | | | | | |
|---|---|---|---|---|---|
| 0 | 1 | 2 | 3 | 4 | 5 |

LEFT EAR (DMSO)

| | | | | | | |
|---|---|---|---|---|---|---|
| average | 25.3 | 26.3 | 28.5 | 30.0 | 31.0 | 31.3 |
| Std. Dev. (SD) | 0.50 | 0.50 | 0.50 | 1.15 | 0.00 | 1.89 |

RIGHT EAR (2% CAFFEINE IN DMSO)

| | | | | | | |
|---|---|---|---|---|---|---|
| average | 25.5 | 26.3 | 28.3 | 29.8 | 32.0 | 33.5 |
| SD | 0.58 | 0.96 | 0.96 | 1.50 | 0.96 | 1.83 |

LEFT EAR (ACETONE)

| | | | | | | |
|---|---|---|---|---|---|---|
| average | 29.7 | 29.7 | 30.0 | 30.0 | 30.0 | 30.0 |
| SD | 0.58 | 0.58 | 0.00 | 0.00 | 0.00 | 0.00 |

RIGHT EAR (1.2% CAFFEINE IN ACETONE)

| | | | | | | |
|---|---|---|---|---|---|---|
| average | 29.7 | 29.7 | 30.0 | 30.0 | 30.0 | 30.0 |
| SD | 0.58 | 0.58 | 0.00 | 0.00 | 0.00 | 0.00 |

In a second experiment, solution was applied to each ear after exposure to UVB irradiation. The following day, the thickness of both ears was measured. UVB irradiation was only applied once and solutions were administered a total of five times (once a day/consecutive five days). The results are provided in Table 2.

TABLE 2

| day | | | | | |
|---|---|---|---|---|---|
| 0 | 1 | 2 | 3 | 4 | 5 |

LEFT EAR (DMSO) + UVB

| | | | | | | |
|---|---|---|---|---|---|---|
| average | 24.30 | 30.30 | 34.80 | 42.50 | 42.80 | 42.50 |
| SD | 0.96 | 2.63 | 4.99 | 6.56 | 5.68 | 5.74 |

RIGHT EAR (2% CAFFEINE IN DMSO) + UVB

| | | | | | | |
|---|---|---|---|---|---|---|
| average | 24.80 | 30.80 | 35.30 | 41.80 | 41.80 | 42.80 |
| SD | 1.26 | 2.75 | 6.08 | 5.25 | 6.65 | 9.57 |

LEFT EAR (ACETONE) + UVB

| | | | | | | |
|---|---|---|---|---|---|---|
| average | 29.00 | 40.00 | 45.70 | 56.00 | 66.00 | 66.00 |
| SD | 1.73 | 2.65 | 2.08 | 2.65 | 3.46 | 6.08 |

RIGHT EAR (1.2% CAFFEINE IN ACETONE) + UVB

| | | | | | | |
|---|---|---|---|---|---|---|
| average | 29.00 | 40.00 | 45.30 | 52.00 | 61.70 | 63.30 |
| SD | 1.00 | 2.65 | 1.50 | 3.00 | 1.53 | 1.53 |

As can be seen from Table 2, ear thickness was increased by UV B irradiation (compare Table 1). Caffeine did not increase ear thickness when compared to solvents (DMSO or acetone). In other words, caffeine did not have primary irritancy at a concentration of 2% in DMSO or 1.2% in acetone.

A third primary irritation experiment was performed. Back skin of mice (FVB strain, female, 7 weeks of age, n=3-4/group) was clipped of hair with an electric clipper and 5 μl of each solution was topically applied. The following day, irritation was evaluated according to five categories (0: no irritation, 1; slight irritation, 2:clear irritation, 3:strong irritation, 4:severe irritation) and 5 μl of each solution was topically applied. This procedure was continued for a total of five consecutive days. The results are provided in Table 3.

TABLE 3

day

| | 0 | 1 | 2 | 3 | 4 | 5 |
|---|---|---|---|---|---|---|
| 2% caffeine in DMSO | 0 | 0 | 0 | 0 | 0 | 0 |
| 0.5% caffeine in DMSO | 0 | 0 | 0 | 0 | 0 | 0 |
| DMSO only | 0 | 0 | 0 | 0 | 0 | 0 |
| 1.2% caffeine in acetone | 0 | 0 | 0 | 0 | 0 | 0 |
| 0.5% caffeine in acetone | 0 | 0 | 0 | 0 | 0 | 0 |
| acetone only | 0 | 0 | 0 | 0 | 0 | 0 |

As seen in Table 3, caffeine did not cause irritation in mouse skin. In other words, caffeine did not have primary irritancy at a concentration of 2% in DMSO and 1.2% in acetone.

### Example 2

### Topical Caffeine Reduces UV-Induced Wrinkling

This experiment was conducted to test the efficacy of caffeine on UVB-induced chronic skin damage. Four groups (group 1: UVB+1.2% caffeine in acetone, group 2: No UVB+1.2% caffeine in acetone, group 3: UVB+acetone, group 4: No UVB and No acetone) of 7-wk-old female hairless Skh-1 mice (n=5/group) were prepared. Mice of groups 1 and 3 were exposed to UVB irradiation, using fluorescent lamps (Southern New England Ultraviolet). The height of the lamps was adjusted to deliver 0.35 mW/cm² at the dorsal skin surface of the mice. Samples were applied (100 μl) on the back of UVB irradiated and non-UVB irradiated mice. This procedure was repeated three times weekly for 10 weeks. UVB irradiation began with a dose of 0.5 minimum erythema dose (MED)(20 mJ/cm²) and gradually increased in increments of 0.5 MED to a maximum dose of 4.5 MED. The total cumulative dose of UVB was 6.54 J/cm².

After 10 weeks, skin wrinkling was evaluated by two independent persons according to five categories (0: no wrinkle; 1: slight wrinkle, 2: clear wrinkle, 3: strong wrinkle; 4: severe wrinkle).

TABLE 4

| group | average | S.D. |
|---|---|---|
| 1: UVB 1.2% + caffeine in acetone | 1.45 | 0.60 |
| 2: No UVB + 1.2% caffeine in acetone | 0 | 0 |
| 3: UVB + acetone | 2.25 | 0.42 |
| 4: No UVB and no acetone | 0 | 0 |

As can be seen in Table 4, caffeine was effective at reducing UVB-induced wrinkle formation at concentrations of 1.2% in acetone.

### Example 3

### Topical Caffeine Reduces UV-Induced Angiogenesis

Mice are sacrificed and back skin samples are snap-frozen in liquid nitrogen. Immunohistochemical stainings will be performed using a monoclonal rat anti-mouse CD31 antibody (Pharmingen). Representative sections will be obtained from UVB-irradiated and non-UVB irradiated mice to be analyzed using a Nikon E-600 microscope (Nikon). Images will be captured with a Spot digital camera (Diagnostic Instruments), and morphometric analyses will be performed using the IP-LAB software. Areas occupied by blood vessels will be determined in the dermis.

### Example 4

### Transgenic Mice Deficient in Replication Checkpoint Show Altered Sensitivity to UV-Induced Aging

To investigate whether topical caffeine works to inhibit wrinkles via ATR, expression constructs of dominant negative ATR and Chk1 proteins were expressed under control of the K14 (skin) promoter in transgenic mice.

All publications, reference, patents, and patent applications cited herein are hereby incorporated by reference. A number of embodiments of the inventions have been described. Nevertheless, it will be understood that various modifications may be made without departing from the spirit and scope of the inventions. Accordingly, other embodiments are within the scope of the following claims.

1. A method of reducing UVB-induced wrinkles in a subject, the method comprising:
identifying a subject having, or at risk for, UVB-induced wrinkles; and
administering to the subject a composition comprising an agent that inhibits ATR mediated signaling, wherein the composition comprises caffeine at a concentration of less than 10%.

2. The method of claim 1, wherein the composition comprises caffeine at a concentration of less than 10%.

3. The method of claim 2, wherein the composition comprises caffeine at a concentration of less than 5%.

4. The method of claim 1, wherein the agent is administered at least twice over a period of one week.

5. The method of claim 1, wherein the agent is formulated as a cosmetic composition.

6. The method of claim 1, wherein the agent is administered prior to UV exposure.

7. The method of claim 1, wherein the agent is administered in combination with one or more cosmetic agent selected from the group consisting of: a sunscreen, a moisturizer, a tanning agent, a fragrance, a makeup foundation.

8. A method of identifying an agent that reduces UVB-induced wrinkles, the method comprising:
evaluating a test agent for the ability to reduce ATR-mediated checkpoint signaling, and
correlating the ability of a test agent that reduces ATR-mediated checkpoint signaling with the agent's ability to reduce UVB-induced wrinkles.

9. The method of claim 8, wherein the test agent is evaluated for the ability to inhibit ATR, ATRIP or CHK1.

10. The method of claim 8, wherein the test agent is a polypeptide, an antibody, a carbohydrate, a lipid, a nucleic acid or a small molecule.

11. The method of claim 8, wherein the test agent is a botanical extract.

12. The method of claim 9, wherein the test agent is evaluating for ability to alter ATR kinase activity or binding to UV damaged DNA.

13. The method of claim 11, wherein the correlating comprises contacting the test agent to a hairless Skh-1 mouse and evaluating wrinkle formation.

14. The method of claim 11, further comprising formulating the test agent as a composition for topical application and, optionally, administering the composition to a subject.

15. A composition suitable for topical application, the composition comprising caffeine in an amount effective to reduce wrinkles when topically applied to a site, the amount being less than 10%.

16. The composition of claim 15 wherein the composition is formulated as a cosmetic.

17. The composition of claim 15 wherein the composition is lotion, salve, gel, or ointment.
